(19) United States
(12) Patent Application Publication
(10) Pub. No.: US 2017/0014275 A1
(43) Pub. Date: Jan. 19, 2017

(54) WOUND CARE DEVICE

(71) Applicant: LOHMANN & RAUSCHER GMBH, Schoenau An Der Triesting (AT)

(72) Inventor: JOHANNES SCHNEIDER, Wien (AT)

(21) Appl. No.: 15/211,241

(22) Filed: Jul. 15, 2016

(30) Foreign Application Priority Data
Jul. 16, 2015 (EP) 15002120.2

(51) Int. Cl. A61F 13/00; A61M 1/00

(52) U.S. Cl. CPC A61F 13/00068; A61M 1/0088

(57) ABSTRACT

In a wound care device having a covering device, the covering device being fixable to the skin surrounding a wound and serving to create a wound space, the covering device having a film tube that is at least in part water-vapor-permeable, and the wound care device having a suction connection by way of which a vacuum in the wound space can be created, an improvement is proposed in which the film tube makes a transition to a retainer flap via a transitional area, at least partly surrounding a sagittal axis extending transversely, in particular approximately perpendicularly, to a frontal plane that is defined by the tube axis and by a radial axis that penetrates the tube axis and extends vertically thereto.

### CROSS-REFERENCE TO RELATED APPLICATIONS

This application is based on and claims priority to European Patent Application Serial No. 15 002 120.2, filed on Jul. 16, 2015, which is incorporated herein by reference in its entirety.

### STATEMENT REGARDING FEDERALLY SPONSORED RESEARCH OR DEVELOPMENT

Not applicable.

### BACKGROUND OF THE INVENTION

The invention relates to a wound care device that has a covering device which can be fixed to the skin surrounding a wound and serves to create a closed-off wound space that contains the wound, the covering device being at least in part water-vapor-permeable and having a film tube.

Such wound care devices are used especially in the context of so-called vacuum therapy. It has been demonstrated that healing of chronic wounds, in particular, can be promoted by applying a vacuum to these wounds. It has furthermore proved advantageous if the wound is covered or filled with an open-pore foam or gauze as a filler material, if the wound is covered in order to create a closed-off wound space that contains the wound and optionally the filler material, and if a suction connection is mounted on the side of the covering device remote from the wound or from the filler material, by way of which suction connection the wound space can be connected to a suction device designed to create a vacuum. In other devices, a flange of the suction connection is covered by the covering device or is received in a pocket surrounding an opening of the covering device. The suction connection can for instance be equipped with a tube that can be connected on one end to a connection device, embodied for instance in the form of a tubular nozzle, of the suction connection and on the other to a suction device. The covering device can be embodied of film-like material, for instance, that is placed in airtight fashion against the skin surface adjacent to the wound.

Wound care devices that can be used in vacuum therapy are described for instance in EP 0 620 720 B1. The disclosure content of this document is expressly incorporated into this specification in terms of the details of the foam-generating means and the suction device that are usable in the context of vacuum therapy. These details may contribute, in combination with the features of the main claim, to attaining the object of this invention. Protection is sought for such combinations as well.

In DE 10 2009 019 646 A1, a contact layer to be placed between the filler material and the base of the wound in order to improve exudate management is described, which forms a drainage space between the filler material and the wound base. The disclosure content of this document is expressly incorporated into this specification in terms of the details of the contact layer forming the drainage space and the wound covering. These details may contribute, in combination with the features of the main claim, to attaining the object of this invention. Protection is sought for such combinations as well.

Suction nozzles that can be used in vacuum therapy and that can be connected via a tube to a suction device are described for instance in WO 03/073970 A1, WO 2008/014358 A2, and WO 2009/124548 A1. A suction connection, called a suction head, with protrusions that serve to guide the flow in the vicinity of the suction connection's boundary face toward the wound is described in EP 1 018 967 B1. In addition, a suction connection with a contact face to be placed against the filler material is disclosed in the form of a disklike shell in EP 1 088 569 B1. In a suction connection described in WO 2010/008167 A2, channels defined by ribs are formed on the boundary face toward the filler material, and through them the wound exudate is meant to be carried toward a suction extraction opening.

In WO 2010/011148 A1, a wound care device that can be used in the context of vacuum therapy is described, which has both a non-permeable tube, which can be pulled over an extremity of the human body, and a perforated body that is to be located between the wound and the tube. With the perforated body, a space is created between the non-permeable tube and the wound base, in which space a vacuum can be created via a tube connection that can be placed in sealing fashion against the non-permeable tube.

In EP 1 162 932 B1, a wound care device is described that has a sheathing of a plastic material and a fluid-absorbent material contained in the sheathing. The wound care device described in this document is intended to protect wounds. For lack of a tube connection, it is unsuitable for use in vacuum therapy.

In EP 2 636 417 A1, a wound care device as defined by the preamble to claim 1 is disclosed. The wound care device can have a water-vapor-permeable film tube, which for treating wounds on the extremities, such as a foot, ankle, arm, or hand, is pulled over the extremity and positioned relative to the wound in such a way that the wound is sealingly covered by the covering device. Next, the covering device can be secured to the skin adjacent to the wound with the adhesive film. For that purpose it may be provided that the adhesive film is drawn from a roll and is wound around one end of the tube in such a way that it sticks to the covering device on the one hand and to the skin on the other. The disclosure content of EP 2 636 417 A1 is expressly incorporated into this specification with regard to the properties of water-vapor-permeable cover films, adhesive films, and with regard to the methods for applying wound care devices. These details may contribute, in combination with the features of the main claim, to attaining the object of this invention. Protection is sought for such combinations as well.

When the wound care device described in EP 2 636 417 A1 is used for wound treatment in the vicinity of the extremities, especially in the vicinity of the upper arm or thigh, it has been found that creating a vacuum in the wound space presents difficulties in many cases.

### BRIEF SUMMARY OF THE INVENTION

In view of these problems in the prior art, it is the object of the invention to disclose a wound care device with which the vacuum therapy can be successfully employed in the vicinity of the upper arms and/or thighs as well.

According to the invention, this object is attained by a refinement of the known wound care device which is essentially characterized in that the film tube makes a transition to a retainer flap via a transitional area, at least partly running around a sagittal axis extending transversely, in particular approximately perpendicularly, to a frontal plane that is defined by the tube axis and by a radial axis that penetrates the tube axis and extends vertically thereto.

The invention traces back to the recognition that the problems observed in the prior art can be definitively ascribed to the lack of sealing in the vicinity of the end of the upper arm or thigh toward the torso when the corresponding extremity is moved, but also to the application and the sealing connection. The transition from the upper arm to the torso is formed by the armpit, which when the arm moves forms creases that impair the sealing connection of the covering device to the skin adjacent to the wound. Similarly, the sealing end of the covering device, in the vicinity of the crotch that forms the transition from the thigh to the torso, is impaired by creasing that develops not only because of a patient's movements but also in the course of applying the device and making the sealing connection.

With wound care devices of the invention, the film tube is widened by a transitional area and a retainer flap, with which the patient's armpit and/or crotch can be superficially covered, so that a sealing connection of the covering device with the patient's skin can be made elsewhere than in these critical areas. Surprisingly, it has been found that the expected impairment of the sealing connection to the patient's skin from the widening of the covering device and the attendant lengthening of the edges of the covering device is so slight that the advantages attained with the invention far outweigh it, and vacuum therapy can be successfully employed in the vicinity of the upper arm and thigh as well; even in the vicinity of the crotch and/or the armpit, vacuum-therapy provisions can be successfully performed using wound care devices of the invention.

In other words, a wound care device of the invention makes closure possible even for body sites that are difficult to seal, such as skin areas that tend to form creases. On the other hand, it is possible to close wounds without causing the skin to stick to adhesive wound bandages.

Within the scope of the invention, a further improvement of the sealing connection of the covering device to the patient's skin can be attained if the retainer flap has a retainer portion that extends at least in part from the transitional area around the critical area of the armpit and/or the crotch toward the remote end of the film tube from the transitional area and that preferably partially runs around a longitudinal axis extending in the frontal plane, in particular running around it over a circumferential angle of 180° or less and 45° or more, and the longitudinal axis is located in a plane that contains the tube axis and extends perpendicular to the sagittal axis.

Because of the widening of the covering device by this kind of retainer portion, a sealing fastening of the retainer portion to body areas of the patient that already have any tendency to creasing when the patient moves, such as in the patient's thigh and/or chest area. On the other hand, because the retainer portion only partially runs around the longitudinal axis, easier application of the covering device to the patient is possible, because the film tube merely has to be pulled over the effected extremity, and it is unnecessary to guide the other extremity through the covering device as well, as would be the case with a covering device in the form of trousers or a jacket.

In wound care devices of the invention, the tube axis (the axis of the extremity to be treated) can extend approximately parallel to the longitudinal axis (the axis of the body). Within the scope of the invention, however, with a view to making the application of the covering device easier, it has proved advantageous if the tube axis and the longitudinal axis form an acute angle of preferably approximately 5° to 45° with one another, in particular 5° to 30°, especially preferably 5° to 15°.

For securely covering critical body areas, such as the armpit or the crotch, it has proved expedient if the edge, oriented toward the film tube, of the transitional area partly runs around the tube axis, preferably over an angular range of 180° or less and more than 20°, in particular more than 45°, and especially preferably more than 60°, and very particularly preferably more than 90°. In this embodiment of the invention, the transitional area can be embodied at least partially as a radially inward segment of a rotary torus.

For reliably covering critical body areas, it has also proved preferable if the transitional area connecting the film tube to the retainer flap runs around the sagittal axis over an angular range of 180° or less and more than 45°, preferably more than 60°, in particular more than 75°.

For a tight connection of the covering device with the skin of the patient without impairment by skin creases that increasingly occur in the vicinity of the armpit and/or the crotch, it has also proved expedient if two retainer flaps extending from the transitional area and spaced apart from one another in the sagittal direction are provided. Then, in treating the arm, one of the retainer flaps can rest on the patient's chest, while the other contacts the patient's back. When a wound in the vicinity of the thigh is being treated, one of the retainer flaps can be located dorsally and the other ventrally.

Preferably, the retainer flaps, on their edges remote from the transitional area, are not connected to one another. Instead, at their ends remote from the transitional areas, they terminate in edges that end in the open. As a result, applying the covering device is easier, because the retainer flaps can be connected to the patient's skin in sealing fashion both dorsally and ventrally, independently of one another.

However, within the scope of the invention it is also conceivable to use embodiments of a kind in which the edges remote from the transitional area are at least in part connected to one another, in particular glued.

A further improvement of the sealing fastening of the covering device to the skin of the patient can be attained if the film tube, on its side remote from the transitional area in the circumferential direction relative to the tube axis, makes a transition to the film tube, on its side remote in the circumferential direction from the transitional area, makes a transition to a collar area circumferentially running around the tube axis over a circumferential angle of 60° or more, preferably 90° or more, in particular approximately 180°-220°. When such a collar area is used, the connection of the covering device to the patient's skin can also be done outside the retainer flap, away from the skin areas that tend to form creases.

To make applying the covering device easier and to improve the sealing properties of the covering device in wound care devices of the invention, it has proved expedient if the collar area merges in the circumferential direction with at least one retainer flap, and the edge of the collar area remote from the film tube is preferably aligned with an edge of the retainer flap that partly runs around the longitudinal axis.

From a manufacturing standpoint and for the sake of simple application, it has proved advantageous if the film tube, the transitional area, the retainer flap, and optionally the collar area all form one continuous three-dimensional structure.

To simplify the sealing connection of the covering device to the patient's skin, this three-dimensional structure can be embodied such that the planar structure is bounded entirely by the remote edge, relative to the transitional area toward the tube axis, of the film tube, by the remote edge relative to the transitional area toward the tube axis, of the collar area, and by edges of the retainer flap or flaps. The sealing connection can then be secured overall to skin areas that have a lesser tendency to form creases.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

Below, the invention is explained in conjunction with the drawings, to which reference is made for all features essential to the invention that are not expressly mentioned in the specification. In the drawing:

FIG. 1 shows a first embodiment of a wound care device of the invention; and

FIG. 2 shows a second embodiment of a wound care device of the invention.

### DETAILED DESCRIPTION OF PREFERRED EMBODIMENT

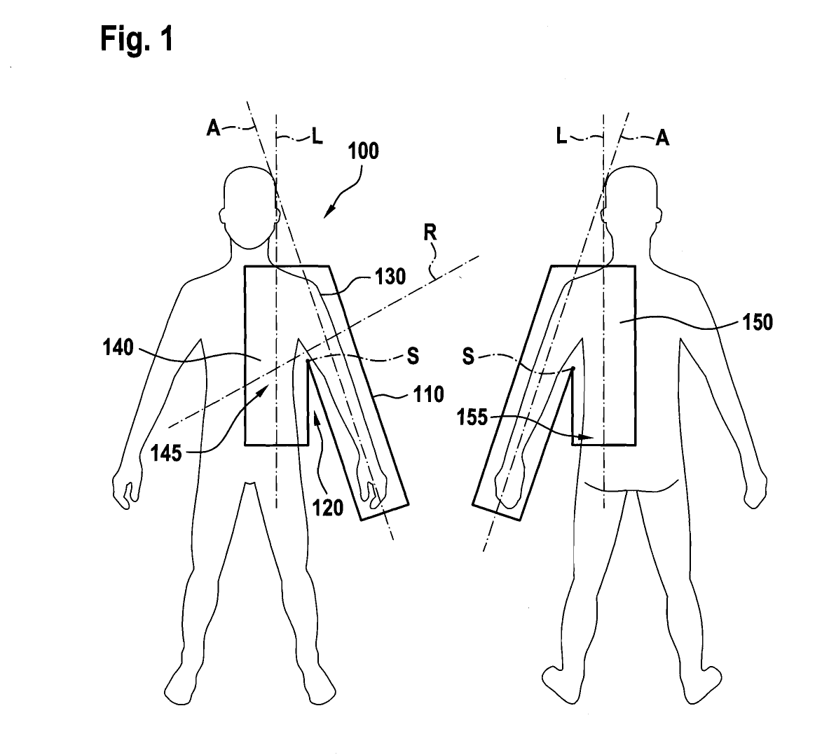

The wound care device 100 shown in FIG. 1 includes a film tube 110, which via a transitional area 120 makes a transition to a ventral retainer flap 140 and a dorsal retainer flap 150. The dorsal retainer flap 150 and the ventral retainer flap 140 are joined to the film tube 110 not only via the transitional area 120 but also via the collar area 130. The film tube 110 is dimensioned such that it can be pulled over the arm of the patient. On its end remote from the patient's hand, the film tube 110 makes a transition to the collar area 130, which runs around the tube axis over an angle of approximately 180° and then makes a transition to the retainer flaps 140 and 150. The ventral retainer flap 140 is bounded rectilinearly and can be connected sealingly to the patient's skin with the aid of suitable adhesive strips or an adhesive applied to the flap itself. The dorsal retainer flap 150 is likewise bounded rectilinearly and can be connected sealingly to the patient's skin via corresponding adhesive strips or an adhesive applied to the retainer flap itself.

On the side remote from the collar area 130 in the circumferential direction with respect to the tube axis, the film tube 110 makes a transition to the transitional area 120. Over a circumferential angle of approximately 320°, this transitional area runs around a sagittal axis S which extends perpendicular to a frontal plane that is defined by the tube axis A and by a radial axis that penetrates the tube axis A and extends perpendicularly to it. Next, the transitional area 120 makes a transition to the retainer flaps 140 and 150, which are joined together on their edge toward the film tube 110, in the area of respective retainer portions 145 and 155 that begin at the transitional area 120 and extend toward the end of the film tube remote from the transitional area 120. On their edges remote from the film tube 110, the ventral retainer flaps 140 and the dorsal retainer flaps 150 end with free edges. All in all, the film tube 110, the collar area 130, the transitional area 120, and the retainer flaps 140 and 150 form one continuous planar structure, which is bounded entirely by the edges of the collar area and of the retainer flaps, so that only in this area does a sealing connection to the patient's skin have to be established. In particular in the vicinity of the armpit, there is no need to establish such a sealing connection, since that area is covered completely and without gaps by the transitional area and the retainer flaps.

The film tube 110 runs completely around a tube axis A in the circumferential direction. Beginning at an edge of the film tube 110 remote from the skin, the transitional area 120 runs around a sagittal axis S over an angular range of approximately 160°. The retainer flaps 140 and 150 run around a longitudinal axis L, which extends in the frontal plane and is located in a plane that is vertical to the sagittal axis S and contains the tube axis A. The retainer flaps 140 and 150 run around the longitudinal axis over a total circumferential angle of only about 180°. The retainer portions 145 and 155 of the retainer flaps 140 and 150 are joined together on their edges toward the film tube 110. Accordingly, the transitional area 120 is joined on the one hand to the film tube 110 over a circumferential angle range of approximately 180° relative to the tube axis A, and on the other to the retainer flaps 140 and 145, respectively, over a circumferential angle range of 180° with respect to the longitudinal axis L.

The suction connection of the wound care device is not shown in FIG. 1. It may be located in the vicinity of the wound area. Normally, it has to be adapted to the particular treatment. Typically, it is placed above the wound, often centrally above the wound, but depending on the location of the wound it can also be in a different position above the wound. Between the wound base and the covering device, expediently also a filler material, such as a filling foam, gauze, or the like, is provided. It can be seen that in the embodiment of the invention shown in FIG. 1, the tube axis and the longitudinal axis that the retainer flaps 140 and 150 run around form an acute angle of approximately 20° with one another.

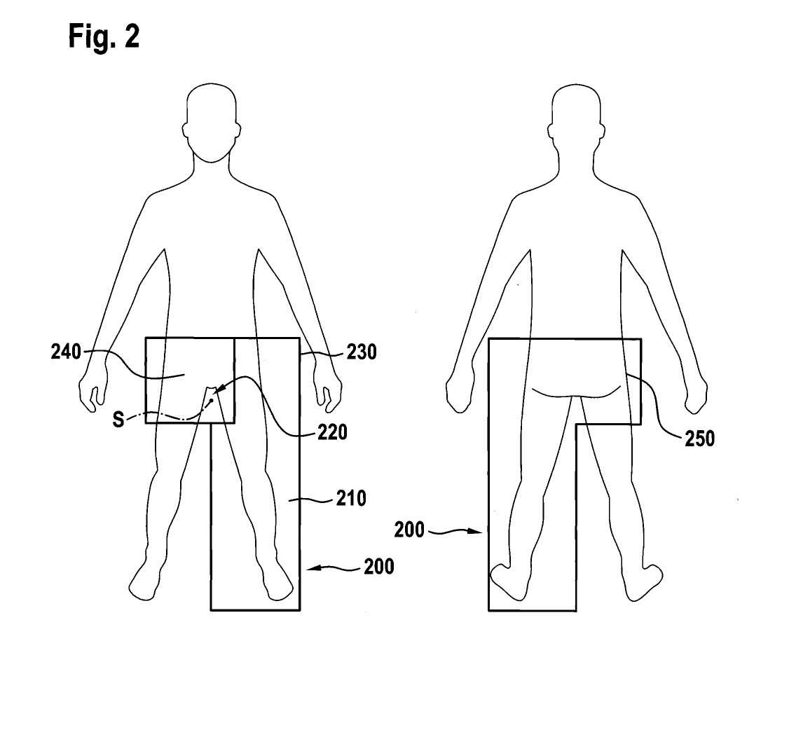

The embodiment of the invention shown in FIG. 2, like the embodiment explained in conjunction with FIG. 1, includes a covering device 200 with a film tube 210, a transitional area 220, a collar area 230, a ventral retainer flap 240, and a dorsal retainer flap 250. The film tube 210 in the embodiment shown in FIG. 2 is dimensioned such that it can be pulled over a patient's leg. The transitional area 220 runs around a sagittal axis S, passing through the crotch of the patient, over a circumferential angle of approximately 180°. The retainer flaps 240 and 250 run around a longitudinal axis that extends parallel to the tube axis. They are joined sealingly to the film tube on its collar area 230 that lengthens the retainer flaps 240 and 250 in the circumferential direction relative to the side remote from the tube axis. The retainer flaps 240 and 250, as in the embodiment of the invention shown in FIG. 1, are bounded rectilinearly and can be connected along their edges sealingly to the patient's skin. The application of the covering device explained in conjunction with FIG. 2 is made easier by the fact that the retainer flaps, on their end remote from the transitional area 220, end in the open, so that the retainer flaps can be connected to the patient's skin independently of one another.

The invention is not limited to the exemplary embodiments explained in conjunction with the drawings. What is essential to attain the object of the invention as described at the outset is that the film tube makes a transition to retainer flaps in at a transitional area, so that not only can areas of the patient's skin that can form creases be sealingly covered, but also there is no need for a direct connection to the patient's skin.

In all the embodiments of the invention, it is also conceivable that the film tube be tightly closed on its axial end remote from the transitional area, so that the foot or hand of the patient can be received entirely in the tube, and no further sealing connection area is necessary in the vicinity of the ankle and/or the wrist.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

1-12. (canceled)

13. A wound care device fixable to the skin surrounding a wound to create a wound space and capable of having a suction connection by way of which a vacuum in the wound space can be created, the wound care device comprising:
a film tube at least part of which is water-vapor-permeable, the film tube having a tube axis;
a retainer flap; and
wherein the film tube makes a transition to the retainer flap via a transitional area at least partly running around a sagittal axis extending transversely and approximately perpendicularly to a frontal plane that is defined by the tube axis and by a radial axis that penetrates the tube axis and extends perpendicularly to it.

14. The wound care device of claim 13, wherein the transitional area runs around the sagittal axis over an angular range of 45° to 180°.

15. The wound care device of claim 13, wherein the retainer flap comprises a retainer portion that extends at least in part from the transitional area toward the end of the film tube remote from the transitional area, the retainer portion partially running around a longitudinal axis extending in the frontal plane.

16. The wound care device of claim 15, wherein the retainer portion runs around the longitudinal axis over a circumferential angle range of 45° to 180°.

17. The wound care device of claim 13, wherein the retainer flap comprises a dorsal retainer flap and a ventral retainer flap.

18. The wound care device of claim 17, wherein each of the dorsal and ventral retainer flaps comprise a retainer portion that extends at least in part from the transitional area toward the end of the film tube remote from the transitional area.

19. The wound care device of claim 18, wherein the retainer portions are joined together on their edges toward the film tube.

20. The wound care device of claim 13, wherein the transitional area further comprises a collar area circumferentially running around the tube axis over a circumferential angle range of 60° to 220°, and wherein the collar area merges with the retainer flap.

21. The wound care device of claim 20, wherein the film tube, the transitional area, the retainer flap, and the collar area all form one continuous three-dimensional structure.

22. The wound care device of claim 21, characterized in that the three-dimensional structure is bounded entirely by the edge of the collar area and by edges of the retainer flap.

23. The wound care device of claim 13, characterized in that the film tube is closed on its end remote from the transitional area.
